(19) United States
(12) Patent Application Publication
(10) Pub. No.: US 2024/0245937 A1
(43) Pub. Date: Jul. 25, 2024

(54) TRANSRECTAL ULTRASOUND PROBE FOR BOILING HISTOTRIPSY ABLATION OF PROSTATE, AND ASSOCIATED SYSTEMS AND METHODS

(71) Applicant: UNIVERSITY OF WASHINGTON, Seattle, WA (US)

(72) Inventors: Vera Khokhlova, Seattle, WA (US); Pavel Rosnitskiy, Seattle, WA (US); Petr V. Yuldashev, Moscow (RU); Tatiana D. Khokhlova, Seattle, WA (US); Oleg A. Sapozhnikov, Seattle, WA (US); George R. Schade, Seattle, WA (US)

(73) Assignee: UNIVERSITY OF WASHINGTON, Seattle, WA (US)

(21) Appl. No.: 18/438,692

(22) Filed: Feb. 12, 2024

Related U.S. Application Data
(63) Continuation of application No. 16/871,980, filed on May 11, 2020, now Pat. No. 11,896,853.
(60) Provisional application No. 62/846,267, filed on May 10, 2019.

(51) Int. Cl. A61N 7/02; A61B 8/00; A61B 8/12; A61B 17/00; A61B 17/32; A61N 7/00

(52) U.S. Cl. CPC A61N 7/022; A61B 8/12; A61B 8/445; A61B 2017/00274; A61B 2017/320069; A61N 2007/0039; A61N 2007/0052; A61N 2007/0078

(57) ABSTRACT

Transrectal ultrasound probe for boiling histotripsy ablation of prostate are presented herein. In one embodiment, a method for a transrectal ultrasound treatment uses high intensity focused ultrasound (HIFU). The method includes: generating a boiling histotripsy (BH) therapy ultrasound by a therapy transducer in a frequency range of 1 MHz to 2.8 MHz and a surface intensity range of 10 W/cm2 to 80 W/cm2. The therapy transducer may be about 50 mm long and about 35 mm wide. The method also includes applying the therapy ultrasound by directing ultrasound pulses having ultrasound shock waves to a target tissue at a focal depth of 2.5 cm to 5.5 cm; generating at least one μm-scale vapor bubble at a target region; growing the at least one vapor bubble to at least one mm-scale bubble; and mechanically disintegrating a surrounding tissue by interactions between mm-scale bubbles and the ultrasound shock waves within a pulse.

### CROSS-REFERENCE TO RELATED APPLICATION

This application is a continuation of U.S. patent application Ser. No. 16/871,980, filed May 11, 2020, which claims the benefit of U.S. Provisional Application No. 62/846,267, filed May 10, 2019, the disclosure of which is expressly incorporated herein by reference in its entirety.

### STATEMENT OF GOVERNMENT LICENSE RIGHTS

This invention was made with government support under R21 CA219793, R01 EB023910, R01 GM122859, and R01 EB007643, awarded by the National Institutes of Health. The government has certain rights to the invention.

### BACKGROUND

Thermal ablation of prostate tissue with high intensity focused ultrasound (HIFU) has recently received FDA approval as a non-invasive treatment alternative to first-line prostate cancer treatment options. However, conventional clinical transrectal HIFU systems have certain limitations. In particular, conventional transrectal HIFU systems may cause collateral damage due to nearfield heating and heat diffusion from the focus. For example, conventional HIFU treatment of a region of prostate generates heat inside the tissue, which then diffuses into other regions of the prostate and the surrounding tissue, potentially causing thermal damage to otherwise healthy tissue. The conventional systems also suffer from insufficient real-time ability to monitor treatment efficacy. Accordingly, systems and methods are needed for improved ablation and imaging of the prostate or other target tissue.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Briefly, the inventive technology uses boiling histotripsy (BH) to cause precise mechanical tissue ablation, thus destroying a target tissue (e.g., cancer tissue). In operation, a sequence of nonlinear ms-long pulses, each having multiple relatively high amplitude shock fronts, heat target tissue up to boiling temperatures within a μm-scale volume during each pulse and causes localized evaporation bubbles at the focus. Thus-generated evaporation bubbles relatively rapidly grow into a mm-scale vapor bubble and cool down. The interaction between the remaining ultrasound shocks of the pulse and the ensuing vapor bubble results in a precise mechanical tissue ablation.

Furthermore, the bubbles generated by BH and the resulting hyperechogenicity of the sonicated tissue allow for reliable real-time targeting and monitoring the treatment with B-mode ultrasound. Moreover, the loss of structure of mechanically ablated tissue results in hypoechogenicity of the treated volume in B-mode images and allows for evaluation of the treatment outcomes. Owing to the rapidity of tissue ablation bioeffects (e.g., on milliseconds scale), low repetition rate of the pulses, and the mechanical mode of action, BH minimizes nearfield heating, heat-sink effects and thermal spread which otherwise complicate conventional HIFU treatments.

In some embodiments, a shaped ultrasound therapy transducer for BH is about 50 mm long and about 35 mm wide, therefore being suitable for a transrectal use. In different embodiments, the therapy transducer may have a focal length of about 40 mm and an aperture of 35×50 mm. The ultrasound therapy transducer may include a central opening for an ultrasound imaging transducer. In some embodiments, the opening is circular having a diameter in a range of 20 mm to 25 mm.

In some embodiments, the therapy ultrasound waves are generated as bursts of waves that are separated by non-emitting periods of time. In some embodiments, the BH pulses have 5 ms duration, 2 Hz pulse repetition frequency, and 20 pulses/focus. These pulses may be delivered to a rectangular grid (6×6 mm with 2 mm spacing) within target tissue using a 1.5 MHz transducer. The surface intensity of the therapy transducer (defined as the power generated per surface area of the transducer) may range from 10 W/cm2 to 80 W/cm2. Based on this level of the surface intensity, the ultrasound shock waves may reach a shock amplitude of about 100 MPa at a focus depth of 40 mm. Such shock amplitude suffices to initiate BH (e.g., to initiate bubble activity). In some embodiments, the therapy transducer operates in a frequency range of 1 MHz to 2.8 MHz.

In one embodiment, a method for a transrectal ultrasound treatment using high intensity focused ultrasound (HIFU) includes: generating a boiling histotripsy (BH) therapy ultrasound by a therapy transducer in a frequency range of 1 MHz to 2.8 MHz and a surface intensity range of 10 W/cm2 to 80 W/cm2, where the therapy transducer is about 50 mm long and about 35 mm wide; applying the therapy ultrasound by directing ultrasound pulses having ultrasound shock waves to a target tissue at a focal depth of 2.5 cm to 5.5 cm; generating at least one μm-scale vapor bubble at a target region; growing the at least one vapor bubble to at least one mm-scale bubble; and mechanically disintegrating a surrounding tissue by interactions between the at least one mm-scale bubble and the ultrasound shock waves within a pulse.

In an embodiment, a focal region for the therapy ultrasound at the target tissue is 0.1 mm to 1 mm wide and 2 mm to 10 mm long.

In another embodiment, a shock amplitude of the therapy ultrasound at a focus depth of 40 mm is about 100 MPa. In an embodiment, a power of the therapy ultrasound is about 200 Watt at the focus depth of 40 mm.

In one embodiment, the therapy transducer is a phased array therapy transducer comprising a plurality of phased array elements.

In an embodiment, the phased array elements are ring-like structures of an annular array. In another embodiment, a phased array therapy transducer comprises 8 phased array elements. In an embodiment, the phased array elements are tile-like structures of a mosaic array.

In one embodiment, the method also includes generating an imaging ultrasound in a frequency range of 7 MHz to 15 MHz by an imaging transducer. In an embodiment, the imaging transducer is placed within a circular hole in the therapy transducer, and wherein a diameter of the hole in the therapy transducer is in a range of 20 mm to 25 mm. In another embodiment, the imaging transducer is configured within a rectangular hole in the therapy transducer, and wherein the hole in the therapy transducer is about 13 mm wide and about 16 mm long.

In one embodiment, a transrectal high intensity focused ultrasound (HIFU) device, includes: a boiling histotripsy (BH) ultrasound probe having a generally rectangular therapy transducer configured to emit therapy ultrasound in an ultrasound frequency range of 1 MHz to 2.8 MHz at a surface acoustic intensity of in a range of 10 W/cm2 to 80 W/cm2, the therapy transducer being about 50 mm long and about 35 mm wide and having a centrally located opening, wherein the therapy transducer is configured to generate shock waves at a focal depth of 2.5 cm to 5.5 cm.

In an embodiment, a focal region of the therapy ultrasound at the target tissue is 0.1 mm to 2 mm wide and 2 mm to 10 mm long.

In another embodiment, the shock waves have an amplitude of the therapy ultrasound of about 100 MPa at a focus depth of 40 mm.

In one embodiment, a power of the therapy ultrasound is about 200 Watt at the focus depth of 40 mm.

In one embodiment, the device also includes: generating an imaging ultrasound in a frequency range of 7 MHz to 15 MHz by an imaging transducer.

In one embodiment, the device also includes an imaging transducer configured to generate an imaging ultrasound in a frequency range of 7 MHz to 15 MHz, where the imaging transducer is configured within a central opening in the therapy transducer, and where the dimensions of the opening are in a range of 15 mm to 25 mm.

In one embodiment, an imaging transducer is configured to generate an imaging ultrasound in a frequency range of 7 MHz to 15 MHz, where the imaging transducer is configured within a rectangular hole in the therapy transducer, and where the hole is about 13 mm wide and about 16 mm long.

In one embodiment, the therapy transducer is a phased array therapy transducer comprising a plurality of phased array elements, and the phased array elements are ring-like structures of an annular array.

In one embodiment, the therapy transducer is a phased array therapy transducer comprising a plurality of phased array elements, and the phased array elements are tile-like structures of a mosaic array.

### DESCRIPTION OF THE DRAWINGS

The foregoing aspects and many of the attendant advantages of this inventive technology will become more readily appreciated as the same become better understood by reference to the following detailed description, when taken in conjunction with the accompanying drawings, wherein:

FIG. 1 is a schematic diagram of a system for transrectal ultrasound boiling histotripsy ablation in accordance with an embodiment of the present technology;

FIG. 2 is a front view of an ultrasound probe in accordance with an embodiment of the present technology;

FIG. 3 is a diagram of an ultrasound field generated by a HIFU transrectal transducer for treating prostate tissue in accordance with an embodiment of the present technology;

FIGS. 4A-4D are different views of a HIFU transrectal therapy transducer in accordance with an embodiment of the present technology;

FIGS. 5A-5D are different views of phased array HIFU transrectal therapy transducers in accordance with embodiments of the present technology;

FIGS. 6A and 6B illustrate ultrasound wave propagation according to prior art and present technology, respectively;

FIG. 7A is a graph of HIFU wave duty cycle in accordance with an embodiment of the present technology;

FIG. 7B illustrates growth of HIFU-caused vapor bubbles in accordance with an embodiment of the present technology;

FIG. 8 is a graph of one cycle of HIFU focal waveform in accordance with an embodiment of the present technology; and

FIGS. 9A and 9B are graphs of one cycle of HIFU focal waveform in accordance with an embodiment of the present technology.

### DETAILED DESCRIPTION

Example devices, methods, and systems are described herein. It should be understood the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood aspects of the present disclosure, as generally described herein, and illustrated in the figures, can be arranged, substituted, combined, separated, and designed in a wide variety of different configurations, all of which are explicitly contemplated herein.

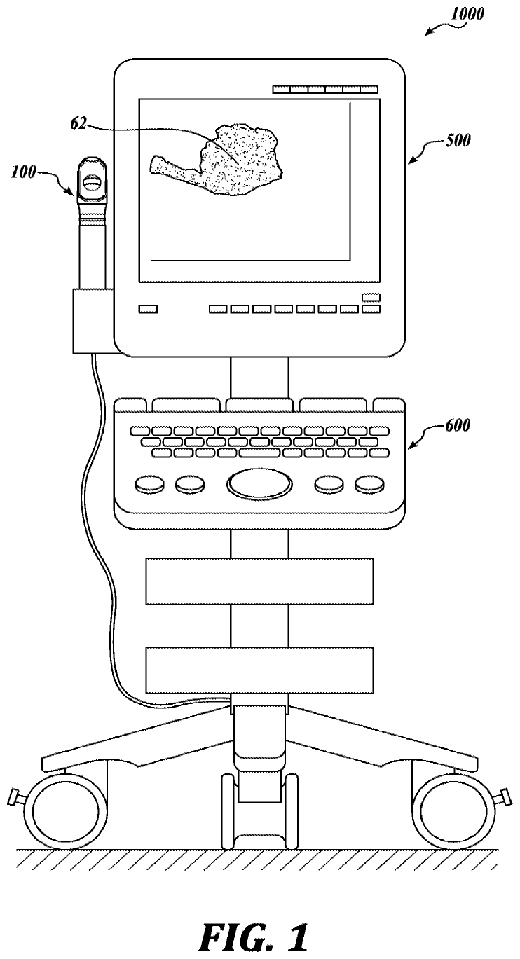

FIG. 1 is a schematic diagram of a system for transrectal ultrasound boiling histotripsy ablation in accordance with an embodiment of the present technology. An ultrasound system 1000 includes an ultrasound probe 100 having a therapy transducer and optionally an imaging transducer. The ultrasound probe 100 may be controlled by a controller (e.g., a computer) 600 having suitable software and commands for controlling the ultrasound. A monitor 500 can display images 62 of the target tissue that are obtained, for example, by an imaging transducer of the ultrasound probe 100 or by a separate imaging device.

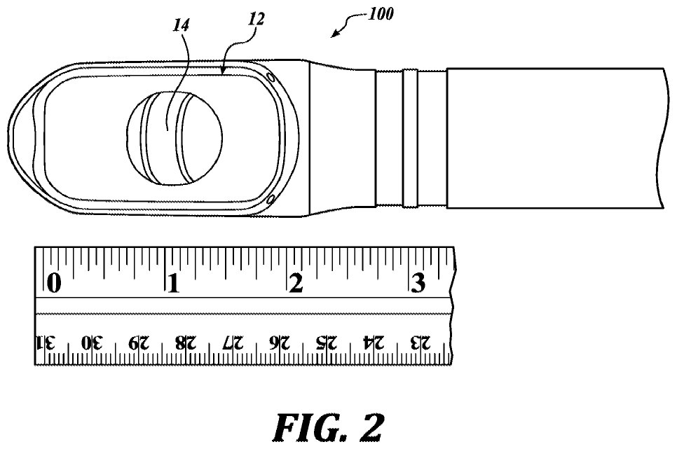

FIG. 2 is a front view of an ultrasound probe 100 in accordance with an embodiment of the present technology. The illustrated ultrasound probe includes a therapy transducer 12 and an imaging transducer 14 that is located in an opening of the therapy transducer. In different embodiments, the imaging transducer may be located away from the therapy transducer 12. In some embodiments, the dimensions of the probe 100 are suitable for transrectal use when treating patient's prostate. For example, the imaging transducer 14 may be about 25 mm long and about 8 mm wide. In some embodiments, the imaging transducer 14 generates imaging ultrasound at a frequency range of 7-15 MHz.

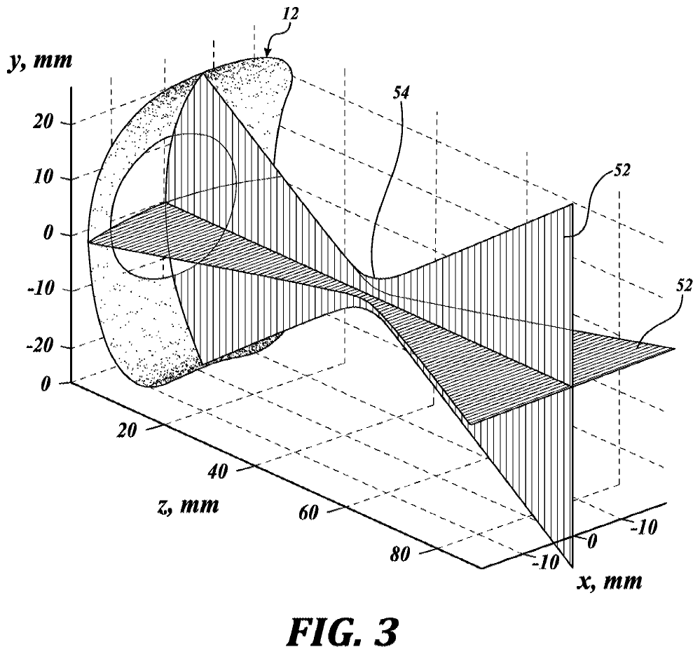

FIG. 3 is a diagram of an ultrasound field generated by a HIFU transrectal transducer for treating prostate tissue in accordance with an embodiment of the present technology. The therapy transducer 12 is located in the X-Y plane of the illustrated 3D coordinated system. The central opening of the therapy transducer 12 may be suitable for holding an imaging transducer. As illustrated in the graph, a sample therapy transducer 12 is about 50 mm long and about 35 mm wide. However, in different embodiments the therapy transducer may have different dimensions.

In operation, therapy transducer 12 generates an ultrasound field 52. In some embodiments, a focal region 54 of the ultrasound field 52 is about 40 mm away from the therapy transducer. The focal area is about 5 mm long and 1 mm wide at low power and linear focusing conditions. For boiling histotripsy conditions, the focal area for the shock amplitude of the pulse is about 2.5 mm by 0.1 mm.

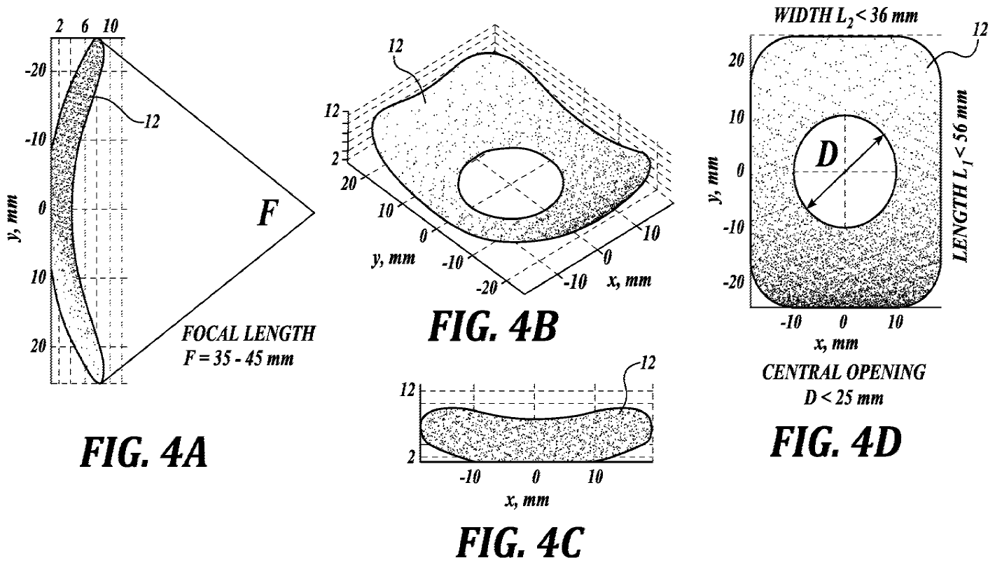

FIGS. 4A-4D are different views of a HIFU transrectal therapy transducer in accordance with an embodiment of the present technology. Illustrated therapy transducer 12 may operate at a frequency range of 1-3 MHz, 1.5-2.8 MHz, 1-2.8 MHz, 2-2.8 MHz, or similar. In some embodiments, the therapy transducer 12 generates ultrasound shocks of about 60-140 MPa in a target tissue located at a focal region about 35-55 mm away from the transducer. In different embodiments, a surface intensity of the therapy transducer (defined as the power generated per unit surface area of the transducer) may range from 10 W/cm2 to 80 W/cm2 or from 10 W/cm2 to 40 W/cm2. In some embodiments, the therapy transducer includes a central opening having a diameter of up to 25 mm. Such central opening may be suitable for an imaging transducer.

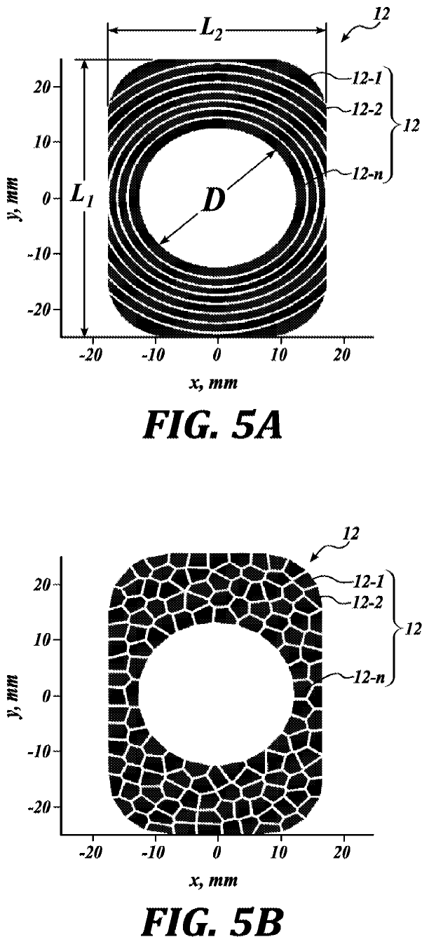

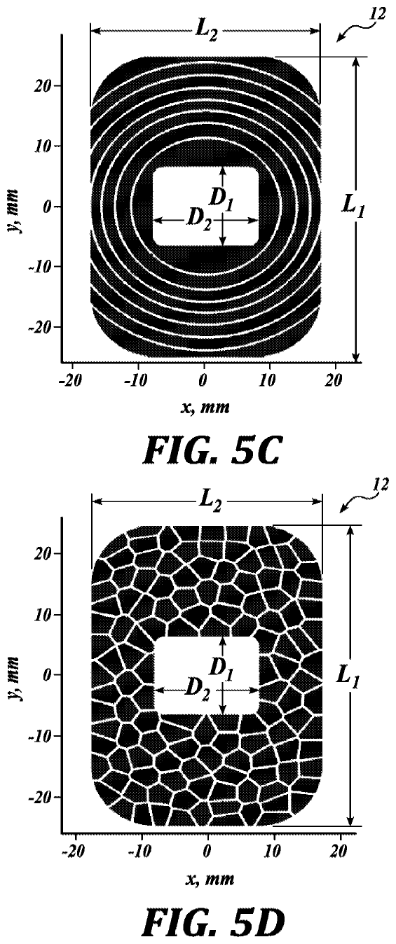

FIGS. 5A-5D are different views of phased array HIFU transrectal therapy transducers in accordance with embodiments of the present technology. In each case, the therapy transducer 12 has a generally rectangular shape with 50 mm length (L₁) and 35 mm width (L₂). A radius of curvature (i.e., the focal length) of the therapy transducer is 40 mm. In other embodiments, other curvatures are possible. In some embodiments, the therapy transducers may have curved edges with about 10 mm radius.

FIG. 5A illustrates a phased array therapy transducer 12 having eight ring-like elements 12-i (also referred to as sections or rings) of equal area. The total area of the illustrated therapy transducer 12 is 1353 mm2. In other embodiments, different number of elements may be used, and the elements may have different areas. For the phased annular array therapy transducer, the focus can be electronically steered along the axis. In some embodiments, the phased array therapy transducer 12 may be controlled to provide a range of steering such that the focal area stays within a region limited by at most 80% decrease of the focal intensity. In different embodiments, other restrictions to steering angle or depth may be implemented. The illustrated therapy transducer has a central opening with a 15-25 mm diameter (D).

FIG. 5B illustrates a phased array therapy transducer 12 having 128 mosaic- or tile-like elements 12-i of equal area. In operation, elements 12-i may be controlled individually or in predetermined groups.

The therapy transducers illustrated in FIGS. 5C and 5D generally correspond to those of FIGS. 5A and 5D except for the rectangular openings for the imaging transducer. In some embodiments, these openings may be 16 mm wide (D₁) and 13 mm high (D₂), leaving a total area of the therapy transducer at 1648 mm2.

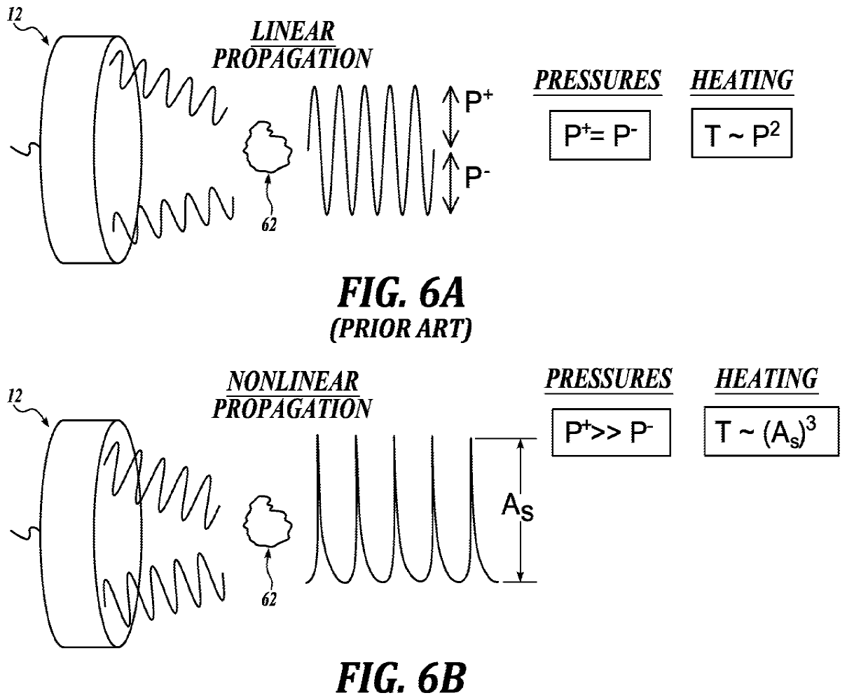

FIGS. 6A and 6B illustrate ultrasound wave propagation according to prior art and present technology, respectively. Both FIGS. 6A and 6B illustrate ultrasound waves generated by therapy transducers 12. The ultrasound waves target tissue 62 (e.g., tumor, blood vessel).

FIG. 6A illustrates the conventional technology that uses smooth waves having generally same peaks of the positive pressure and negative pressure (P⁺ and P⁻). The conventional technology has certain shortcomings. For example, the smooth ultrasound waves slowly heat tissue and, when applied over a period of time, the heat diffuses to tissues that surrounds the target tissue at the focus. In many applications, such heating must be minimized or controlled by, for example, by limiting the exposure time or energy of the therapy ultrasound. In general, with conventional smooth wave technology a heat generated by the ultrasound scales with square of pressure peaks of the ultrasound (P²).

FIG. 6B illustrates an embodiment of the inventive technology. With the inventive technology, the emitted smooth therapy ultrasound waves develop into nonlinear shock waves as they propagate through the tissue. These nonlinear shock waves are characterized by their positive pressure (P⁺) being significantly higher than the negative pressure (P⁻). Furthermore, ultrasound shock waves heat the target tissue relatively fast, since the heat generation scales with the ultrasound shock wave amplitude to third power (As³). As a result, a boiling temperature may be reached relatively fast, for example within several milliseconds, thus limiting the undesired heating of the surrounding tissue. The vapor bubble initiated by the localized μm scale boiling relatively quickly grows into larger vapor cavity and cools down while expanding from the hot focus. In some embodiments, relatively small vapor bubble having μm-scale may grow into mm-scale bubble within milliseconds. The remaining shock wavefronts within the same pulse keep interacting with these bubbles. As explained above, interaction of upcoming shocks with large scale bubble mechanically destroy (ablate) target tissue.

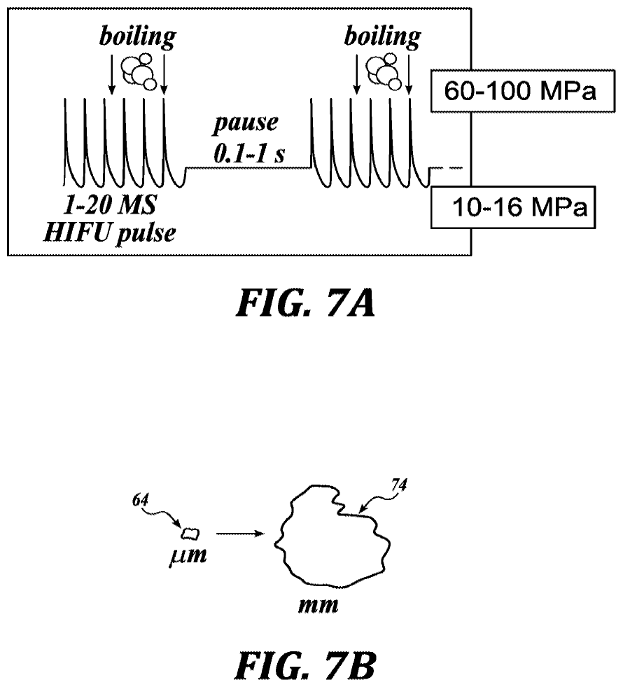

FIG. 7A is a graph of HIFU pressure pulses in accordance with an embodiment of the present technology. In some embodiments, the pulses of HIFU may extends over 1-20 ms, followed by a pause of 0.1-2 second. As explained above, the ultrasound shock waves within a given pulse may cause localized boiling in the target tissue, therefore generating vapor bubbles at the focal area. Next, these vapor bubbles may rapidly grow into significantly larger vapor bubble during the same pulse in the HIFU operation. These bubbles interact with remaining shock wavefronts within the same pulse. An example of such rapid growth in the bubble size is illustrated in FIG. 7B, which shows an initially generated vapor bubble 64 at μm-scale growing into a mm-scale gas bubble 74.

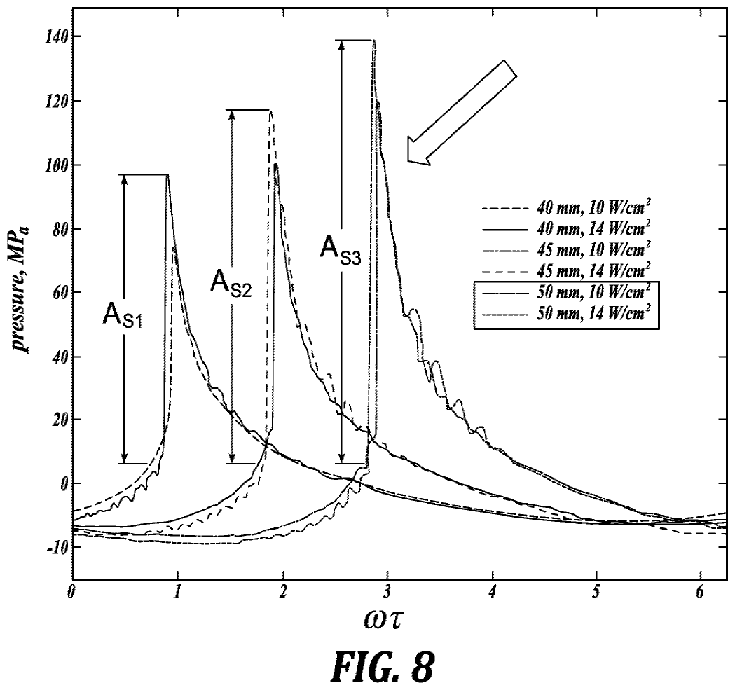

FIG. 8 is a graph of one cycle of HIFU focal waveforms in accordance with an embodiment of the present technology. The horizontal axis represents time elapsed, and the vertical axis represents pressure at the target area. FIG. 8 shows modeled focal waveforms in tissue produced by the HIFU probe 100. As explained above, the HIFU system 100 can generate ultrasound shock waveforms in the focal region of the therapy transducer. In some embodiments, the pressure amplitudes of shock waveforms (As) may range from about 60 MPa to about 140 MPa. In general, the pressure amplitudes of the shock waveforms scale up with higher surface intensity and longer length of the therapy transducer. For example, for a focal length of 40 mm, probe length of 40 mm and surface intensity of 14 W/cm2, the pressure amplitude Aₛ₁ is about 95 MPa. For a higher value of the probe length of 50 mm and the same surface intensity of 14 W/m2, the pressure amplitude Aₛ₃ is about 140 MPa, at least in part because for transducers with larger focusing angle, the focal lobe is shorter, the ultrasound shock waves have a shorter distance to develop thus requiring higher pressures. Other values of illustrated parameters are also possible in different embodiments.

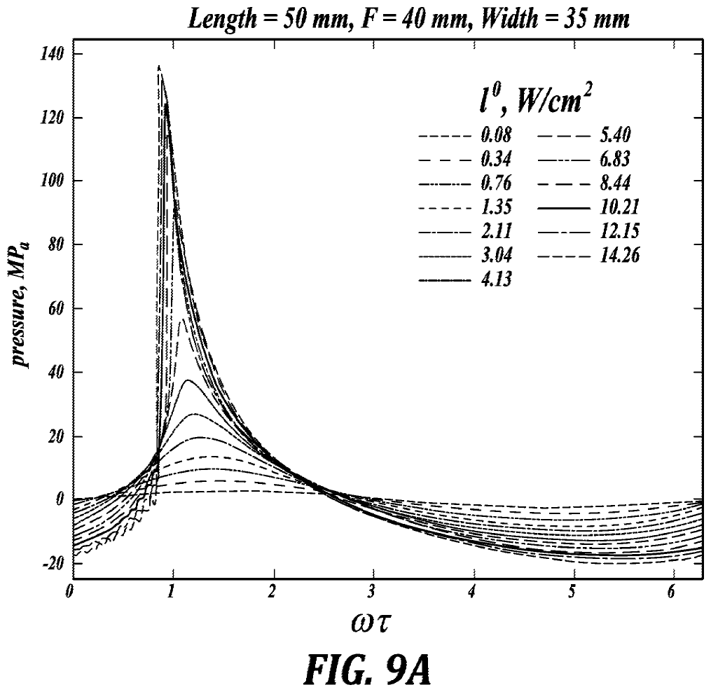

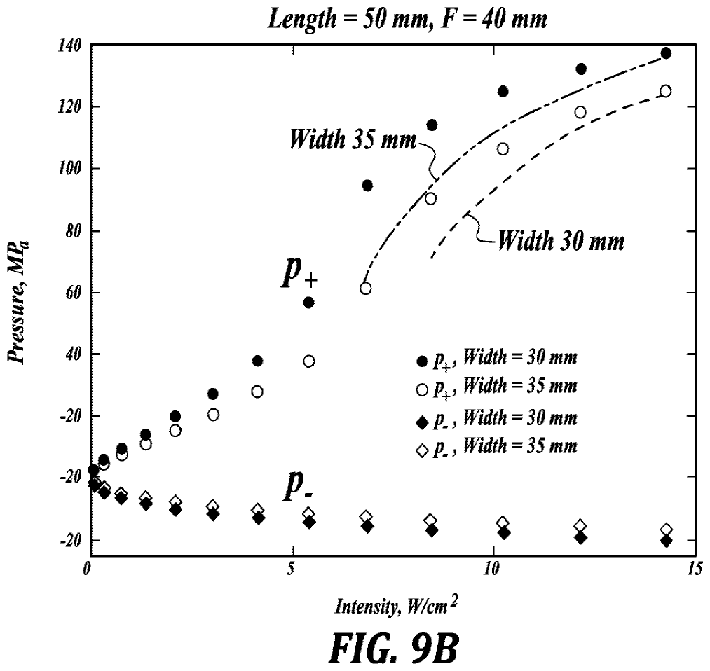

FIG. 9A is a graph of one cycle of HIFU focal waveforms in accordance with an embodiment of the present technology. The horizontal axis represents time elapsed, and the vertical axis represents pressure at the target area. The graph shows modeled pressure results in water for a therapy probe having 50 mm length and 35 mm width, at the focal length of 40 mm. Different curves in the graph represent surface intensity of the modeled probe in a range of 0.08 W/cm2 to 14.26 W/cm2. The resultant ultrasound pressures at the focus scale up with the increased surface intensity. Furthermore, for the modeled probe, the shock waves develop only after a certain threshold value of the surface intensity is reached, which in this case is about 10 W/cm2. In other embodiments, other values of the parameters may apply.

FIG. 9B is a graph of HIFU peak positive and peak negative pressures of the focal waveform in accordance with an embodiment of the present technology. The horizontal axis represents surface intensity of the therapy probe, and the vertical axis represents peak pressures at the target area. The graph shows modeled pressure results for a therapy probe having 50 mm length and the focal length of 40 mm. Two therapy probes are modeled: one having a width of 30 mm (solid symbols) and another having a width of 35 mm (open symbols). Based on the simulation results, the positive peak pressure (P⁺) and the resultant ultrasound shock amplitude increases with increased probe width.

Many embodiments of the technology described above may take the form of computer- or controller-executable instructions, including routines executed by a programmable computer or controller. Those skilled in the relevant art will appreciate that the technology can be practiced on computer/controller systems other than those shown and described above. The technology can be embodied in a special-purpose computer, controller or data processor that is specifically programmed, configured or constructed to perform one or more of the computer-executable instructions described above. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like).

From the foregoing, it will be appreciated that specific embodiments of the technology have been described herein for purposes of illustration, but that various modifications may be made without deviating from the disclosure. For example, in some embodiments the counter or controller may be based on a low-power buck regulator connected to a capacitor. Moreover, while various advantages and features associated with certain embodiments have been described above in the context of those embodiments, other embodiments may also exhibit such advantages and/or features, and not all embodiments need necessarily exhibit such advantages and/or features to fall within the scope of the technology. Accordingly, the disclosure can encompass other embodiments not expressly shown or described herein.

The present application may also reference quantities and numbers. Unless specifically stated, such quantities and numbers are not to be considered restrictive, but exemplary of the possible quantities or numbers associated with the present application. Also, in this regard, the present application may use the term “plurality” to reference a quantity or number. In this regard, the term “plurality” is meant to be any number that is more than one, for example, two, three, four, five, etc. The terms “about,” “approximately,” etc., mean plus or minus 5% of the stated value.

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

What is claimed is:

1. An apparatus, comprising:
an ultrasound probe having a therapy transducer configured to emit ultrasound pulses including ultrasound waves having a frequency between 1 MHz and 2.8 MHz, the therapy transducer configured to have a surface intensity between 10 W/cm² and 80 W/cm², the ultrasound waves emitted by the therapy transducer being configured to generate shock waves that induce a boiling histotripsy in a target tissue of a patient,
wherein the ultrasound probe is configured for insertion into a body lumen of the patient and be positionable proximate to the target tissue, and
wherein the therapy transducer includes a plurality of transducer elements arranged in a mosaic-like pattern around a centrally disposed opening, the plurality of transducer elements including multiple groups of transducer elements that are individually controllable to generate the shock waves.

2. The apparatus of claim 1, wherein the therapy transducer is configured for inducing the boiling histotripsy at a focal depth between 2.5 cm and 5.5 cm.

3. The apparatus of claim 1, wherein the ultrasound probe further includes an imaging transducer configured to emit ultrasound waves having a frequency between 7 MHz and 15 MHz for imaging the target tissue.

4. The apparatus of claim 3, wherein the imaging transducer is disposed within the centrally disposed opening.

5. The apparatus of claim 4, wherein the centrally disposed opening is a rectangular opening.

6. The apparatus of claim 5, wherein the centrally disposed opening has dimensions between 13 mm and 16 mm.

7. The apparatus of claim 1, wherein the therapy transducer has dimensions between 35 mm and 50 mm.

8. The apparatus of claim 1, wherein the shock waves generated by the therapy transducer are configured for inducing the boiling histotripsy by:
generating, within each ultrasound pulse, at least one μm-scale vapor bubble at a target region;
growing the at least one vapor bubble to at least one mm-scale bubble; and
mechanically disintegrating a surrounding tissue by interactions between the at least one mm-scale bubble and the shock waves within the ultrasound pulse.

9. The apparatus of claim 1, wherein the therapy transducer is configured for inducing the boiling histotripsy in a focal region in the target tissue, the focal region having a width of between 0.1 mm to 1 mm and a length of between 2 mm and 10 mm.

10. The apparatus of claim 1, wherein the therapy transducer is configured to emit the ultrasound pulses that generate the shock waves having an amplitude of between about 60 MPa and 140 Mpa at a distance of about 35 and 55 mm away from the therapy transducer.

11. The apparatus of claim 1, wherein the ultrasound probe is a transrectal probe configured to be inserted into a rectum of the patient.

12. The apparatus of claim 1, wherein the plurality of transducer elements are disposed on a curved surface.

13. The apparatus of claim 1, wherein the therapy transducer has a curved edge.

14. An apparatus, comprising:
an ultrasound probe including:
a therapy transducer configured to emit ultrasound pulses including ultrasound waves having a frequency between 1 MHz and 2.8 MHz, the therapy transducer configured to have a surface intensity between 10 W/cm² and 80 W/cm², the ultrasound waves transmitted by the therapy transducer being configured to generate shock waves that induce a boiling histotripsy in a target tissue of a patient, the therapy transducer including a plurality of transducer elements being arranged around a rectangular opening; and
an imaging transducer disposed within the rectangular opening, the imaging transducer configured to emit ultrasound waves having a frequency between 7 MHz and 15 MHz,
wherein the ultrasound probe is configured for insertion into a body lumen of the patient and be positionable proximate to the target tissue.

15. The apparatus of claim 14, wherein the therapy transducer is configured for inducing the boiling histotripsy at a focal depth between 2.5 cm and 5.5 cm.

16. The apparatus of claim 14, wherein the rectangular opening has dimensions between 13 mm and 16 mm.

17. The apparatus of claim 14, wherein the therapy transducer has dimensions between 35 mm and 50 mm.

18. The apparatus of claim 14, wherein the shock waves generated by the therapy transducer are configured for inducing the boiling histotripsy by:
generating, within each ultrasound pulse, at least one μm-scale vapor bubble at a target region;
growing the at least one vapor bubble to at least one mm-scale bubble; and
mechanically disintegrating a surrounding tissue by interactions between the at least one mm-scale bubble and the shock waves within the ultrasound pulse.

19. The apparatus of claim 14, wherein the therapy transducer is configured for inducing the boiling histotripsy in a focal region in the target tissue, the focal region having a width of between 0.1 mm and 1 mm and a length of between 2 mm and 10 mm.

20. The apparatus of claim 14, wherein the therapy transducer is configured to emit the ultrasound pulses that generate the shock waves having an amplitude of between about 60 MPa and 140 MPa at a distance of about 35 and 55 mm away from the therapy transducer.

21. The apparatus of claim 14, wherein the ultrasound probe is a transrectal probe configured to be inserted into a rectum of the patient.

22. The apparatus of claim 14, wherein each of the plurality of transducer elements has an equal area.

23. The apparatus of claim 14, wherein the plurality of transducer elements includes multiple groups of transducer elements that are individually controllable for generating the shock waves.

24. The apparatus of claim 14, wherein the plurality of transducer elements are disposed on a curved surface.

25. The apparatus of claim 14, wherein the therapy transducer has a curved edge.
